(19) United States
(12) Patent Application Publication
(10) Pub. No.: US 2018/0055384 A1
(43) Pub. Date: Mar. 1, 2018

(54) SYSTEM AND METHOD FOR NON-INVASIVE HEALTH MONITORING

(71) Applicant: RIOT SOLUTIONS PVT LTD., Kerala (IN)

(72) Inventors: Aardra Kannan Ambili, Kerala (IN); Kannan Natarajan, Kerala (IN)

(73) Assignee: RIOT SOLUTIONS PVT LTD., Kerala (IN)

(21) Appl. No.: 15/688,150

(22) Filed: Aug. 28, 2017

(30) Foreign Application Priority Data
Aug. 26, 2016 (IN) 201641029076

(51) Int. Cl. A61B 5/0205; A61B 5/08; G06F 19/00; A61B 5/01

(52) U.S. Cl. CPC A61B 5/02055; A61B 5/0803; A61B 5/72; A61B 5/01; G06F 19/3418

(57) ABSTRACT

Systems and methods are provided for monitoring a patient's health is provided. The system includes a monitoring device configured to monitor one or more health parameters of the patient, wherein the monitoring device includes a sensing unit configured to receive the signals pertaining to the one or more health parameters, wherein the sensing unit includes one or more sensors configured to receive signals through non-physical contact with the patient. The system further includes a memory configured to store data pertaining to the signals, and a processor configured to analyze the data using pre-determined information and programmed artificial intelligence.

### CLAIM OF PRIORITY

This application claims priority to Indian Patent Application No. 201641029076, filed Aug. 26, 2016, herein referenced in its entirety.

### FIELD OF THE EMBODIMENTS

The present invention is generally related to monitoring devices. The present invention is particularly related to health monitoring devices. The present invention is more particularly related to non-invasive, non-contact health monitoring devices.

### BACKGROUND OF THE EMBODIMENTS

Health monitoring systems are continuously being developed in recent years. More specifically, these devices continuously monitor the health of patients based on physiological parameters. The demand for health monitoring devices and systems is increasing due to the rapid development in this field of technology, increased health awareness, increasing health costs, aging population, and the like. To address this demand, a variety of prototypes and commercial products have been developed in the course of recent years which aim at providing real-time feedback information about the patient's health condition, either to the user himself or to a medical center or to a supervising professional physician, in case of possible health threatening conditions.

The conventional health monitoring systems are mostly invasive and/or require contact with the human body and may pose problems to the patients while using them. Even the conventional non-invasive health monitoring systems require the user to be in contact with the device such as a strip, sock, bed, etc., which may be uncomfortable to the user. Hence, there is a need for a system and method for non-invasive non-contact health monitoring. Further, there is a need for a system and method for alerting the patient and a caregiver using the non-contact non-invasive health monitoring system. Furthermore, there is a need for a method and system for monitoring and analyzing patient data using artificial intelligence.

The above-mentioned shortcomings, disadvantages, and problems are addressed herein and which will be understood by reading and studying the following specification.

### SUMMARY OF THE EMBODIMENTS

Any electronic device that is in contact with an infant's body has a safety risk, as most battery powered devices have Li ion batteries. According to an aspect of the present invention, a non-contact non-invasive solution is presented that is capable of remotely collecting accurate respiration and motion data enables the running of artificial intelligence algorithms that will analyze the data and warn caregivers and emergency response teams of any irregularities. Without constant monitoring, there is a significant chance that users who are predisposed with this condition can have an attack that could be fatal. The present invention cuts the time that occurs between monitoring the patient to notifying the caregivers of potential life-threatening attacks/irregularities, and this is exceedingly crucial in saving lives.

According to an aspect of the present invention, a system for monitoring a patient's health is provided. The system includes a monitoring device configured to monitor one or more health parameters of the patient, wherein the monitoring device includes a sensing unit configured to receive the signals pertaining to the one or more health parameters, wherein the sensing unit includes one or more sensors configured to receive signals through non-physical contact with the patient. The system further includes a memory configured to store data pertaining to the signals, and a processor configured to analyze the data using pre-determined information and programmed artificial intelligence.

According to another aspect of the present invention, a method for monitoring a patient's health is provided. The method includes receiving, using a sensing unit coupled to a monitoring device, one or more signals pertaining to one or more health parameters of the patient, wherein the sensing unit includes one or more sensors, analyzing, using a processor, data pertaining to the one or more signals, the analyzing including determining one or more medical conditions of the patient using pre-determined information and programmed artificial intelligence, and transmitting, to a remote server, data analyzed using the processor.

It is an object of the present invention to provide the system for monitoring a patient's health, wherein the one or more health parameters are selected from the group consisting of: respiration patterns; heart rate; temperature; and sleep patterns.

It is an object of the present invention to provide the system for monitoring a patient's health, wherein the monitoring device is a wall mounted device.

It is an object of the present invention to provide the system for monitoring a patient's health, wherein the monitoring device is further configured to transmit and receive one or more electromagnetic signals.

It is an object of the present invention to provide the system for monitoring a patient's health, wherein the one or more sensors are selected from the group consisting of: an ultrasound reader; a camera; a video recorder; and a microphone.

It is an object of the present invention to provide the system for monitoring a patient's health, wherein the system further includes a data transceiver configured to receive data from the sensing unit and transmit the data to a wireless server.

It is an object of the present invention to provide the system for monitoring a patient's health, wherein the system further includes a mobile electronic device configured to receive data analyzed by the processor.

It is an object of the present invention to provide the system for monitoring a patient's health, wherein the mobile electronic device further includes a graphical user interface configured to enable a user to access data stored in the memory.

It is an object of the present invention to provide the system for monitoring a patient's health, wherein the processor is further configured to send one or more alerts to the mobile electronic device if a pre-determined medical condition if met based on the analyzed data.

It is an object of the present invention to provide the method for monitoring a patient's health, wherein the analyzing further includes incorporating analytics, thresholding, filtering, noise reduction, digital signal processing algorithms, and artificial intelligence algorithms.

It is an object of the present invention to provide the method for monitoring a patient's health, wherein the one or more health parameters are selected from the group consisting of: respiration patterns; and sleep patterns.

It is an object of the present invention to provide the method for monitoring a patient's health, wherein the monitoring device is a wall mounted device.

It is an object of the present invention to provide the method for monitoring a patient's health, wherein the one or more sensors are selected from the group consisting of: an ultra-wide band radar wave sensor; an air quality sensor; an ultrasound reader; a camera; a video recorder; and a microphone.

It is an object of the present invention to provide the method for monitoring a patient's health, wherein the method further includes transmitting, to a mobile electronic device or a network of devices, data analyzed by the processor.

It is an object of the present invention to provide the method for monitoring a patient's health, wherein the method further includes transmitting, to a mobile electronic device, data analyzed in the cloud.

It is an object of the present invention to provide the method for monitoring a patient's health, wherein the method further includes accessing the data analyzed by the processor, using a graphical user interface coupled to the mobile electronic device.

It is an object of the present invention to provide the method for monitoring a patient's health, wherein the method further includes sending an alert to a user if a pre-determined medical condition is met based on the data analyzed by the processor.

### BRIEF DESCRIPTION OF THE DRAWINGS

The other objects, features, and advantages will occur to those skilled in the art from the following description of the preferred embodiment and the accompanying drawings in which:

FIG. 1 shows a system diagram illustrating a non-invasive, non-contact health monitoring environment, according to an embodiment of the present invention.

FIG. 2 shows a perspective view of a wall mounting device for providing a non-invasive, non-contact health monitoring environment, according to an embodiment of the present invention.

FIG. 3 show's a sectional side view of the wall mounting device, according to an embodiment of the present invention.

Although the specific features of the present invention are shown in some drawings and not in others. This is done for convenience only as each feature may be combined with any or all of the other features in accordance with the present invention.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

The various embodiments of the present invention provide systems and methods for continuously monitoring the health of a user. The system includes a monitoring device that monitors the health parameters of patients using respiration, body movement, and sleep patterns. According to an embodiment, the monitoring device monitors heart rate, temperature, and/or any other suitable biometric data. According to an embodiment of the present invention, the monitoring device is a wall mounted device that is capable of transmission and reception of electromagnetic signals. The wall mounted device includes a sensing unit to receive a respiration and motion signals of the patients under observation. According to an embodiment, the sensing unit includes one or more sensors. According to an embodiment, a sensing unit, located in the wall mounted device, senses the respiration and motion of the patient under observation and analyzes respiration and motion patterns using signal processing algorithms and artificial intelligence algorithms.

According to an embodiment, the data collected from the sensing unit is continuously uploaded to the cloud. According to an embodiment, the data collected from the sensing device is processed in two ways. The processing unit on the wall mounted device processes the data from the sensing unit using filtering algorithms, thresholding algorithms, noise reduction, digital signal processing algorithms, and/or artificial intelligence algorithms. The cloud is also configured to support processing of data, more specifically, thresholding and artificial intelligence algorithms. The algorithms in both cases produce actionable insights for the caregiver in the form of visualizations/recommendations on the communication device of the caregiver, patient, doctor, and/or any other relevant party.

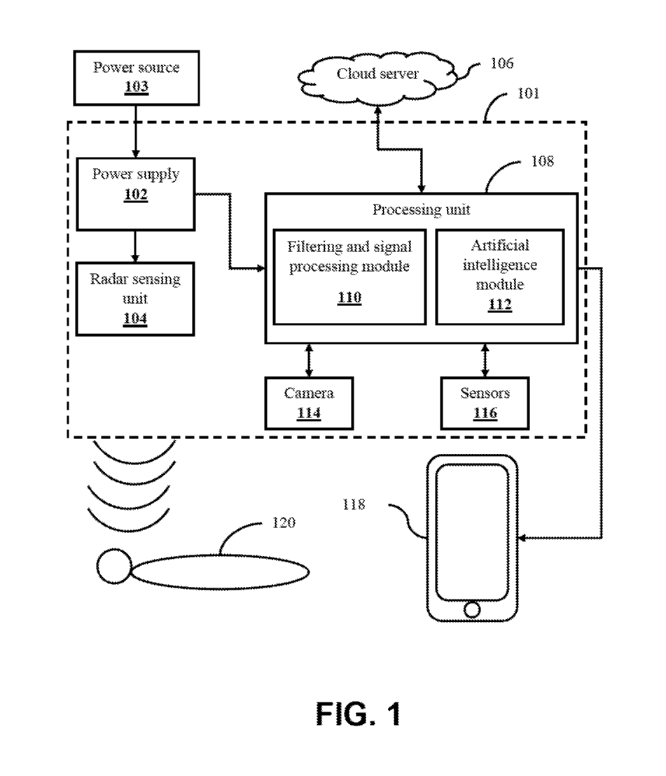

Referring now to FIG. 1, a system diagram illustrating a non-invasive, non-contact health monitoring environment is illustratively depicted, in accordance with an embodiment of the present invention.

According to an embodiment, the system diagram includes a wall-mounted device 101, a monitoring device 118, a cloud server 106, a power source 103, and a patient under observation 120. According to an embodiment, the wall mounted device 101 includes a power supply module 102, a radar sensing unit 104, a processing unit 108, a filtering and signaling module 110, an artificial intelligence module 112, a camera 114, and a sensor module 116. The power supply 102 receives external power from an external power source 103, such as an AC power adapter.

According to an embodiment, the patient under observation 120 is a subject whose respiration and motion patterns are monitored using the sensing unit 104.

According to an embodiment, the power supply 102 provides power to the radar sensing unit 104 for detecting the respiration and sleep pattern and also to the processing unit 108 for processing the information required for analyzing the patients. The power supply 102 also includes an alternative power source such as a backup Battery which will be used as an alternate power source in the event of power outages.

The radar sensing unit 104 is part of the wall-mounted device 1001 for receiving the respiration and motion signals of the patient under observation 120. The radar sensing unit 104 is preferably placed in close proximity to the patient under observation 120. According to an embodiment of the present invention, the distance is not more than 5 m, to ensure that the patient under observation 120 is in a 60-degree cone of the sensing system. Further, the radar sensing unit 104 includes a number of components that has the capability to filter the primary noise while receiving the signals of respiration and motion.

The radar sensing unit 104 sends the received physiological data to the processing unit 108. According to an embodiment of the present invention, the processing unit 108 is a part of the wall mounted device 1001. According to an embodiment of the present invention, the processing unit 108 is located inside the wall mounting device. The processing unit 108 processes the received physiological data from the radar sensing unit 104. The processing unit 108 includes the filtering and signal processing module 110 and the artificial intelligence module 112. The filtering and signal processing module 110 filters the unnecessary signals transmitted by the radar sensing unit 104 and processes the signals to derive the pattern of respiration and motion. Further, the combination of the derived pattern is combined and is compared with a set of predetermined patterns. Further, according to an embodiment, the artificial intelligence module 112 uses artificial intelligence algorithms to derive patterns and recognize the abnormalities in the condition of the patient under observation 120.

The processing unit 108 receives sensing data from the camera 114 and the sensor module 116. The camera 114 is configured for providing visuals of the patient under observation. The sensor module 116 includes one or more sensors that determine temperature of the patient, sound, air quality, humidity and temperature of the room. The examples of the sensors used in the sensor module 116 include, but are not limited to, a temperature sensor, a humidity sensor, acoustic sensor, and the like. According to an embodiment, the sensor module 116 includes an ultrasound reader, an ultra-wide band radar wave sensor, an air quality sensor, a camera, a video recorder, a microphone 225, and/or any other suitable sensor. According to an embodiment, the camera takes images and/or videos of the patient and sends the images and/or video to a secondary device, such as a mobile electronic device 118.

According to an embodiment, the cloud server 106 stores data collected from the wall mounted device and related information of the patients. According to an embodiment, the cloud server 106 is configured such that it is capable of performing processing of the data to analyze and derive information about the patients under observation. According to an embodiment of the present invention, the processing unit also transmits the processed signals to the cloud server 106 for future reference.

According to an embodiment of the present invention, whenever an abnormality in the patient's condition is detected, an alarm is raised and a notification is sent to an authorized caregiver through the monitoring device 118. The examples of the monitoring device 118 include, but are not limited to a smartphone, a laptop, a wearable device, a smart television, a tablet computer, and the like. The examples of the authorized caregivers include, but are not limited to a concerned physician, a family member, a nurse, a hospital helpline number, a friend, a third-party professional, and the like. The notification for the caregiver is sent in a plurality of ways, including but not limited to an SMS, a call, an alert warning through a mobile application, an internet messaging, and the like. According to an embodiment, the notification is sent if a pre-determined health condition is met.

According to an embodiment of the present invention, the processing unit 108 combines the respiration and motion patterns of the patients under observation to determine the early signs of a plurality of diseases and syndromes. The examples of the diseases and syndromes detected include, but are not limited to sleep apnea, epilepsy, insomnia, heart rate, seizure, cardiac arrest, sudden infant death syndrome (SIDS), respiration patterns, movement patterns, and the like.

According to an embodiment, the notification for the caregiver is sent by means of different communication networks. The examples of the communication network include, but are not limited to the Internet, an intranet, a radio-frequency network, telephonic network, a local area network (LAN), a wide area network (WAN), a proximity network such as Bluetooth, Wi-Fi, ZigBee, Bluetooth Low Energy (BLE), and the like.

According to an embodiment, the present system may incorporate presence detection, determining, e.g., intrusion detection, the presence of one or more individuals in a room, and/or any other suitable presence detection. According to an embodiment, the present system may incorporate medical technology and be configured for use in hospitals and/or other medical locations for use in medical diagnoses. Data may be sent to a nurse's (or other medical professional) station, where the nurse (or other medical professional) could be monitoring the vital signs of one or more patients. According to an embodiment, the analyzed data may be sent to a doctor (or other medical professional) in the form of a comprehensive health report.

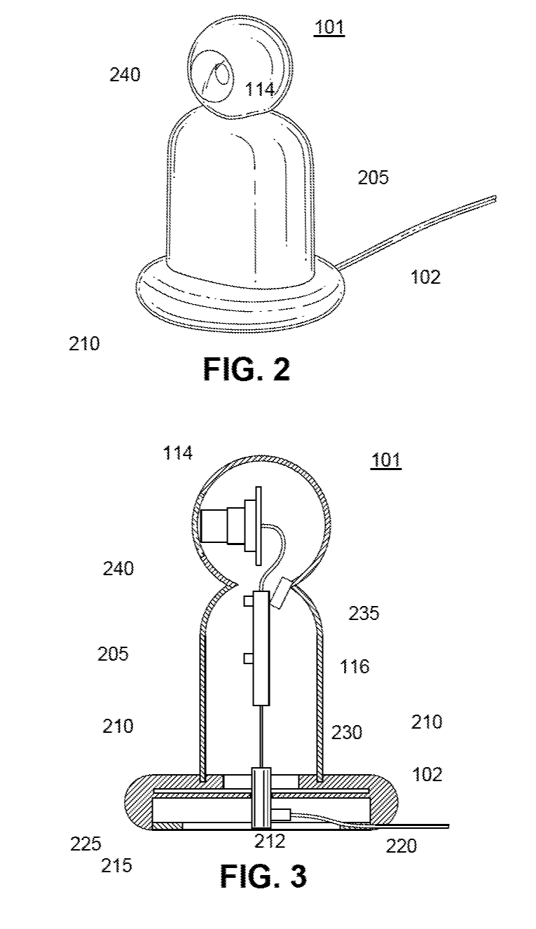

According to an embodiment, the present system may be used in the diagnosis of sleep apnea or other sleep related conditions. According to an embodiment, the present system may be used in seizure detection, epilepsy monitoring, monitoring for asthma and/or other respiratory issues, and/or the detection and/or monitoring of any other suitable ailments. Referring now to FIGS. 2-3, a perspective view (FIG. 2) and a sectional side view (FIG. 3) of the wall mounting device 101 are illustratively depicted, in accordance with an embodiment of the present invention.

According to an embodiment, the wall mounting device 101 includes an outer housing 205. According to an embodiment, the outer housing 205 includes a base section 210. The base section 210 may include one or more securing mechanisms 212 configured to secure the wall mounting device 101 to a surface.

According to an embodiment, the wall mounting device 101 further includes a processor 215, a memory 220, a microphone 225, a speaker 230, an orientation mechanism 235, one or more sensors 116, and/or a camera 114. It is noted, however, that the wall mounting device 101 may further include any other suitable elements for the performance of the wall mounting device 101. According to an embodiment, a transparent covering 240 is located in front of the lens of the camera 116. According to an embodiment, the wall mounting device 101 may further include a power supply 102.

According to an embodiment, the orientation mechanism 235 aids in determining the position of the wall mounted device 101.

According to an embodiment, the processor 215 may perform one or more of the functions of the wall mounted device 101 as described herein.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modifications.

When introducing elements of the present disclosure or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A system for monitoring a patient's health, comprising:
a monitoring device configured to monitor one or more health parameters of the patient, the monitoring device including:
a sensing unit configured to receive the signals pertaining to the one or more health parameters, wherein the sensing unit includes one or more sensors configured to receive signals through non-physical contact with the patient;
a memory configured to store data pertaining to the signals; and
a processor configured to analyze the data using pre-determined information and programmed artificial intelligence.

2. The system as recited in claim 1, wherein the one or more health parameters are selected from the group consisting of: respiration patterns; heart rate; temperature; and sleep patterns.

3. The system as recited in claim 1, wherein the monitoring device is a wall mounted device.

4. The system as recited in claim 1, wherein the monitoring device is further configured to transmit and receive one or more electromagnetic signals.

5. The system as recited in claim 1, wherein the one or more sensors are selected from the group consisting of: an ultrasound reader; a camera; a video recorder; and a microphone.

6. The system as recited in claim 1, further comprising a data transceiver configured to receive data from the sensing unit and transmit the data to a wireless server.

7. The system as recited in claim 1, further comprising a mobile electronic device configured to receive data analyzed by the processor.

8. The system as recited in claim 7, wherein the mobile electronic device further includes a graphical user interface configured to enable a user to access data stored in the memory.

9. The system as recited in claim 7, wherein the processor is further configured to send one or more alerts to the mobile electronic device if a pre-determined medical condition if met based on the analyzed data.

10. A method for monitoring a patient's health, comprising:
receiving, using a sensing unit coupled to a monitoring device, one or more signals pertaining to one or more health parameters of the patient, wherein the sensing unit includes one or more sensors;
analyzing, using a processor, data pertaining to the one or more signals, the analyzing including determining one or more medical conditions of the patient using pre-determined information and programmed artificial intelligence; and
transmitting, to a remote server, data analyzed using the processor.

11. The method as recited in claim 10, wherein the analyzing further includes incorporating analytics, thresholding, filtering, noise reduction, digital signal processing algorithms, and artificial intelligence algorithms.

12. The method as recited in claim 10, wherein the one or more health parameters are selected from the group consisting of: movement patterns; respiration patterns; heart rate; temperature; and sleep patterns.

13. The method as recited in claim 10, wherein the monitoring device is a wall mounted device.

14. The method as recited in claim 10, wherein the one or more sensors are selected from the group consisting of: an ultrasound reader; an ultra-wide band radar wave sensor; an air quality sensor; a camera; a video recorder; and a microphone.

15. The method as recited in claim 10, further comprising transmitting, to a mobile electronic device or a network of devices, data analyzed by the processor.

16. The method as recited in claim 15, further comprising accessing the data analyzed by the processor, using a graphical user interface coupled to the mobile electronic device.

17. The method as recited in claim 15, further comprising sending an alert to a user if a pre-determined medical condition is met based on the data analyzed by the processor.

18. The method as recited in claim 15, further comprising transmitting, to a mobile electronic device, data analyzed using cloud computing.
